(19) United States
(12) Patent Application Publication
(10) Pub. No.: US 2023/0037325 A1
(43) Pub. Date: Feb. 9, 2023

(54) COMPOSITIONS AND METHODS FOR TREATING WOUNDS IN LIVESTOCK

(71) Applicant: Juliane E. Baish, Las Cruces, NM (US)

(72) Inventor: Juliane E. Baish, Las Cruces, NM (US)

(21) Appl. No.: 17/394,044

(22) Filed: Aug. 4, 2021

(51) Int. Cl. A61K 35/644; A61K 33/38; A61K 36/889; A61K 38/39; A61K 36/9066; A61K 38/48

(52) U.S. Cl. CPC A61K 35/644; A61K 33/38; A61K 36/889; A61K 36/9066; A61K 38/39; A61K 38/4873; C12Y 304/22004

(57) ABSTRACT

The present disclosure relates in general to compositions and methods for treating wounds in livestock that help wounds heal faster with less visible scarring. One aspect of the present disclosure includes compositions for treating wounds in livestock that utilize natural, non-toxic and organic ingredients such as honey, colloidal silver, coconut oil, collagen peptides, turmeric, sugar, salt and bromelain. Another aspect of the present disclosure includes methods for treating wounds in livestock utilizing the composition through means of a topical ointment or spray. The intended purpose of the present disclosure is to treat wounds in livestock by reducing inflammation and healing scratches, strangles and flesh wounds, including bacterial, viral and fungal infections.

### FIELD OF THE INVENTION

The present disclosure relates to compositions and methods for treating wounds in livestock. More specifically, the purpose of the invention is to provide compositions for treating wounds in livestock that comprise natural, non-toxic and organic ingredients that help wounds heal faster with less visible scarring. Applications of the compositions of the present disclosure include methods for treating wounds in livestock, such as scratches, strangles and flesh wounds, including bacterial, viral and fungal infections, through means of a topical ointment or spray.

### BACKGROUND OF THE INVENTION

Wound management is a part of everyday life for livestock owners and farm managers. Horses, cattle, dairy, sheep, swine, poultry and other domesticated animals become subject to different types of wounds or injuries depending on their environment, population levels and seasonal conditions. How the wound was created and its specific location on the animal are significant factors that influence the type of treatment available and subsequent risk of infection. Additional factors in the context of parasite control during the summer months may also influence how the wound is treated to prevent cutaneous myiasis. Myiasis is a condition where flies lay eggs in a wound or injury and the hatching larvae feed on the host tissues causing extreme damage and infection.

The healing of any wound is influenced most by its initial management. After stabilizing the animal, first aid should be applied as soon as safely possible. This may include the application of direct pressure to control any active bleeding. Wound lavage is next initiated to irrigate the wound, wash away both visible and microscopic particles, and to reduce bacterial load in the damaged tissue. Debridement may also be needed to remove segments of tissue that have questionable viability. Depending on the severity and type of injury, an open wound may be closed with sutures, staples or cyanoacrylate to avoid complications. When a wound cannot be closed, open wound management may be appropriate. Such wounds may include those where there has been a loss of skin or its particular location on an extremity makes closure unamenable. Traditional open wound management typically includes repeated bandaging until the wound heals (e.g., at least once daily and often twice daily in the early stages of healing). Use of such methods consequently increases cost for the livestock owner and prolongs the time for healing. Wound contracture may further complicate matters by resulting in a loss of joint function or range of motion due to scarring. Cosmetics may also be an issue with open wound management whereas open wounds tend to result in a hairless scar. This may be unacceptable to a livestock owner where cosmetics are important depending on the intended use of the animal, for example, if the injured animal is to be featured in livestock shows or competitions.

To assist with the healing process and to help reduce scarring, antibiotics and other types of medicinal ointments are often utilized in open wound management. Broad-spectrum bactericidal antibiotics, e.g., a first-generation cephalosporin, are generally recommended. Larvicides may also be utilized in dressings to kill maggots of parasitic flies and to prevent myiasis. Given concerns sometimes associated with the overuse of antibiotics in the livestock industry, in particular, that antibiotic resistance in humans may be promoted by the widespread use of antibiotics in animals, several countries have thus restricted antibiotic use in animal agriculture. Some health conscious consumers also prefer to buy meat raised without antibiotics or pesticides. Consequently, there is a growing demand for antibiotic-free and organic meat products. Livestock owners, and specifically horse owners, may also prefer natural, non-toxic ingredients when treating their valuable animals.

For these reasons a desire remains to provide compositions and methods for treating wounds in livestock that comprise natural, non-toxic and organic ingredients. What is further needed are compositions and methods for treating wounds in livestock that reduce inflammation, fight against viral, bacterial and fungal infections, and help wounds heal from the inside out with less visible scarring.

### SUMMARY OF THE INVENTION

According to one aspect of the present disclosure, a composition for treating wounds in livestock is provided. The composition includes natural, non-toxic and organic ingredients. In particular, the ingredients may comprise or consist of honey, colloidal silver, coconut oil, collagen peptides, turmeric, sugar, salt and bromelain. The ingredients may further comprise or consist of the following weight percentages: honey at approximately 29 wt. %; colloidal silver at approximately 19 wt. %; coconut oil at approximately 18 wt. %; collagen peptides at approximately 10 wt. %; turmeric at approximately 12 wt. %; sugar at approximately 5 wt. %; salt at approximately 5 wt. %; and bromelain at approximately 2 wt. %. Specifically, the colloidal silver may be at approximately 100 - 150 ppm. The composition may be adapted to be utilized as a topical ointment or a topical spray to treat different types of livestock wounds located on a variety of places on an injured animal.

According to another aspect of the present disclosure, a method of treating wounds in livestock is provided. The method may include preparing a composition for treating wounds in livestock, the composition including natural, non-toxic and organic ingredients. In particular, the ingredients may comprise or consist of honey, colloidal silver, coconut oil, collagen peptides, turmeric, sugar, salt and bromelain. The ingredients may further comprise or consist of the following weight percentages: honey at approximately 29 wt. %; colloidal silver at approximately 19 wt. %; coconut oil at approximately 18 wt. %; collagen peptides at approximately 10 wt. %; turmeric at approximately 12 wt. %; sugar at approximately 5 wt. %; salt at approximately 5 wt. %; and bromelain at approximately 2 wt. %. Specifically, the colloidal silver may be at approximately 100 - 150 ppm. The method may include adding the ingredients into a mixing vessel wherein the ingredients are thoroughly mixed for approximately 15 - 25 minutes to form the composition. The method may further comprise applying the composition to a livestock wound as a topical ointment or as a topical spray to effectively treat the wound.

### Principal Objects and Advantages of the Invention

Therefore, it is a principal object, feature, and/or advantage of the present disclosure to overcome the aforementioned deficiencies in the art and provide compositions and methods for treating wounds in livestock that comprise natural, non-toxic and organic ingredients.

Another object, feature, and/or advantage of the present disclosure is to provide compositions and methods for treating wounds in livestock that help wounds heal faster with less visible scarring.

Yet another object, feature, and/or advantage of the present disclosure is to provide compositions and methods for treating wounds in livestock that reduce inflammation and heal scratches, strangles and flesh wounds, including bacterial, viral and fungal infections.

A further object, feature, and/or advantage of the present disclosure is to provide compositions and methods for treating wounds in livestock that may be utilized on a diverse population of livestock encompassing varying species, breeds and ages.

A still further object, feature, and/or advantage of the present disclosure is to provide compositions and methods for treating wounds in livestock that may be conveniently applied through means of a topical ointment or spray to wounds that may be located on a variety of places on the injured animal, including harder-to-bandage or reach areas.

Other objects, features, and advantages of this disclosure will become apparent from the following description taken in conjunction with the accompanying drawing wherein are set forth, by way of illustration and example and without limitation, certain aspects of this disclosure. The present disclosure is not to be limited to or by these objects, features, and advantages. No single aspect need provide each and every object, feature, or advantage.

### BRIEF DESCRIPTION OF THE DRAWINGS

FIG. 1 is a flow diagram showing a method for treating wounds in livestock utilizing a composition in accordance with aspects of the present disclosure.

### DETAILED DESCRIPTION OF THE INVENTION

The present disclosure is directed towards compositions and methods for treating wounds in livestock. It is contemplated that the compositions and methods of the present disclosure may be used with a diverse population of livestock encompassing varying species, breeds and ages. Non-limiting examples of livestock contemplated by the present disclosure may include horses, cattle, dairy, sheep, swine, poultry and other domesticated animals. It is further contemplated that the compositions and methods of the present disclosure may be utilized to treat wounds in livestock that include, but are not to be limited to, scratches, strangles and flesh wounds, including bacterial, viral and fungal infections. Effective treatment may include reducing inflammation, helping wounds heal faster as compared to non-treatment, and healing wounds from the inside out with minimal scarring.

While certain aspects of the present disclosure are shown and described herein, it is understood that such aspects are merely exemplary. The present disclosure is not intended to be limited to these specific aspects and may encompass other aspects or embodiments. Therefore, specific compositional and process details disclosed herein are not to be interpreted or inferred as limiting, but merely as a basis for the claims and as a representative basis for teaching one skilled in the art how to make and use the disclosed subject matter.

It must further be noted that the singular terms “a,” “an,” and “the” as used herein may include plural referents unless the context clearly dictates otherwise. As used herein, in particular aspects, the terms “about” or “approximately” when preceding a numerical value indicates the value plus or minus a range of 0.2 %, 0.4 %, 0.6 %, 0.8 % or 1 %. In other aspects, the terms “about” or “approximately” when preceding a numerical value indicates the value plus or minus a range of 2 %, 3 %, 4 % or 5 %. In yet other aspects, the terms “about” or “approximately” when preceding a numerical value indicates the value plus or minus a range of 6 %, 7 %, 8 %, 9 % or 10 %. Furthermore the transitional phrase “comprising” that is synonymous with “including,” “containing,” and “characterized by” as used herein is inclusive or open-ended and does not exclude additional, unrecited elements, steps or ingredients. Alternatively the transitional phrase “consisting of” as used herein is closed and excludes any element, step or ingredient not specified.

In one aspect of the present disclosure, there is provided a composition 10 for treating wounds in livestock that comprises natural, non-toxic and organic ingredients. The specific formulation for the composition 10 is shown in Table 1.

Table 1

Composition for treating wounds in livestock

| Ingredient | Fluid Ounce | Weight Percentage (approx.) |
|---|---|---|
| Honey | 384 oz. | 29 % |
| Colloidal Silver | 256 oz. | 19 % |
| Coconut Oil | 240 oz. | 18 % |
| Collagen Peptides | 128 oz. | 10 % |
| Organic Turmeric | 160 oz. | 12 % |
| Organic Sugar | 64 oz. | 5 % |
| Organic Salt | 64 oz. | 5 % |
| Bromelain | 32 oz. | 2 % |

In particular, the honey ingredient of the composition 10 may comprise organic, raw honey. Topical application of the honey to livestock wounds provides beneficial healing effects that are a result of the honey’s high osmolality, low pH and hydrogen peroxide activity. Methylglyoxal concentrations in honey also provide significant antibacterial properties. Notably, Manuka honey native to New Zealand has antiviral, anti-inflammatory and antioxidant benefits in addition to its antibacterial effects.

The colloidal silver ingredient of the composition 10 may comprise certified, pure colloidal silver at approximately 100 - 150 ppm. In some aspects, the silver ingredient may comprise certified, pure colloidal silver at approximately 120 ppm. The primary benefits of topical silver to livestock wounds are its antimicrobial and anti-inflammatory effects. Such benefits of colloidal silver application help skin wounds heal faster, lower the risk of infection, and aid in strengthening the immune response.

The coconut oil (Cocos nucifera) ingredient of the composition 10 may comprise organic, unrefined coconut oil or butter. Topical application of the coconut oil to livestock wounds assists with healing by improving antioxidant status and increasing levels of collagen in the skin. Coconut oil also has antimicrobial properties that inhibit the growth of the bacteria Staphylococcus aureus causing staph infections and the yeast Candida albican, a common source of yeast infections.

The collagen peptides ingredient of the composition 10 may comprise hydrolyzed, collagen peptides derived from animal sources. Topical application of the collagen peptides to livestock wounds accelerates the wound healing process by supporting re-epithelialization, angiogenesis, tissue regeneration and increased collagen deposition to the wound bed. The use of collagen peptides also help minimize the effects of scarring by supporting the animal's immune response to close the wound and generate new tissue around the damaged area.

The turmeric ingredient of the composition 10 may comprise organic turmeric (Curcuma longa). In some aspects the composition 10 may comprise turmeric extracts that mostly contain the compound curcumin, the main active ingredient in turmeric. The primary benefits of topical turmeric to livestock wounds, and particularly turmeric’s main active compound curcumin, include anti-inflammatory, anti-oxidant, anti-carcinogenic, anti-mutagenic, anti-coagulant and anti-infective effects. The use of turmeric also enhances granulation tissue formation, collagen deposition, tissue remodeling and wound contraction to hasten healing and reduce scarring.

The sugar ingredient of the composition 10 may comprise organic sugar. Topical application of the sugar to livestock wounds provides beneficial healing effects that are a result of the sugar’s high osmolality which draws fluid out of the wound and inhibits the growth of bacteria. The use of sugar also helps control odor and infection while preserving viable tissue and aiding in debridement of damaged tissue.

The salt ingredient of the composition 10 may comprise organic salt or an organic saline solution. Topical application of the salt to livestock wounds provides a powerful microbial inhibitor that promotes healing and anti-inflammation through a process called osmosis. For example when a bacterium cell has a lower concentration of salt than its surrounding environment, water passes out of the bacterium into the environment, eventually dehydrating the bacterial cell and killing it. The use of salt thus helps control bacterial infections in livestock wounds.

The bromelain ingredient of the composition 10 may comprise organic bromelain, a natural mixture of enzymes found in pineapples (Ananas comosus). Primary benefits of topical bromelain to livestock wounds include the reduction of inflammation and assistance with the debridement of dead tissue to promote healing.

In some aspects of the present disclosure, the composition 10 may comprise the aforementioned ingredients and concentrations recited in Table 1 along with additional, unrecited ingredients. In other aspects of the present disclosure, the composition 10 may consist of the aforementioned ingredients and concentrations recited in Table 1 and exclude any additional, unrecited ingredients.

In one non-limiting example, the composition 10 may consist of the aforementioned ingredients at the specific fluid ounces recited in Table 1 to produce 1,328 total fluid ounces of the composition 10. The composition 10 may thereafter be deposited into 110, 12 oz. containers and applied as a topical ointment to treat wounds in livestock. Alternatively the composition 10 may be deposited into 83, 16 oz. spray bottles and applied as a topical spray to treat wounds in livestock. The composition 10 may be conveniently applied through means of the topical ointment or spray to wounds that may located on a variety of places on the injured animal, including hard-to-bandage areas.

In another non-limiting example, the composition 10 may consist of the aforementioned ingredients at the specific weight percentages recited in Table 1 to produce a total desired amount of the composition 10. The composition 10 may thereafter be deposited into container(s) and applied as a topical ointment to treat wounds in livestock. Alternatively the composition 10 may be deposited into spray bottle(s) and applied as a topical spray to treat wounds in livestock. The composition 10 may be conveniently applied through means of the topical ointment or spray to wounds that may be located on a variety of places on the injured animal, including harder-to-bandage or reach areas.

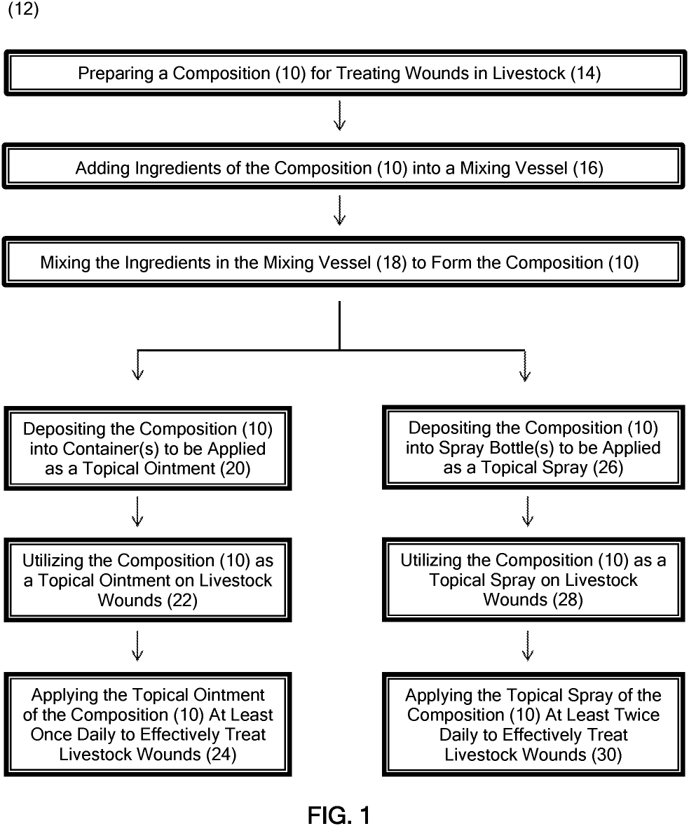

FIG. 1 illustrates a flow diagram showing another aspect of the present disclosure, a method 12 for treating wounds in livestock utilizing the composition 10. In particular, the method 12 may include preparing 14 the composition 10. The composition 10 may be prepared 14 by adding 16 the ingredients recited in Table 1 into a mixing vessel. The mixing vessel may comprise a cylindrical shape having a contoured bottom (e.g., tank, pot or bowl). The mixing vessel may be further comprised of stainless steel, metal or plastic materials having a non-stick coating to help provide adequate stirring and mixing of the ingredients without leaving residue on the interior of the mixing vessel. The ingredients of the composition 10 may be added 16 into the mixing vessel consecutively in the order recited in Table 1. Alternatively, the ingredients of the composition 10 may be added 16 into the mixing vessel in any order.

In one non-limiting example, the ingredients of the composition 10 may be added 16 into the mixing vessel at the specific fluid ounces recited in Table 1 to produce 1,328 total fluid ounces of the composition 10. In another non-limiting example, the ingredients of the composition 10 may be added 16 into the mixing vessel at the specific weight percentages recited in Table 1 to produce a total desired amount of the composition 10. In some aspects of the present disclosure only the ingredients and amounts/concentrations recited in Table 1 are added 16 into the mixing vessel to consist of the composition 10. In other aspects, additional ingredients and amounts/concentrations unrecited in Table 1 may be added 16 into the mixing vessel to comprise the composition 10.

Shown in FIG. 1, the method 12 may include mixing 18 the ingredients in the mixing vessel using an agitator or rotating impeller to form the composition 10. The agitator or rotating impeller imparts motion and shear within the mixing vessel to induce a thorough mixing 18 of the ingredients. The ingredients should be thoroughly mixed 18 in the mixing vessel by the agitator or rotating impeller for approximately 15 - 25 minutes. In some aspects of the present disclosure the ingredients are thoroughly mixed in the mixing vessel by the agitator or rotating impeller for approximately 20 minutes. The mixing 18 may be accomplished manually (e.g., by hand) or mechanically (e.g., using a mixer). The mixing 18 of the ingredients influences the quality and efficiency of the composition 10 and therefore should be maintained until the composition 10 appears to have a uniform consistency.

Further shown in FIG. 1, an aspect of the method 12 of the present disclosure may include depositing 20 the composition 10 into container(s) to be applied as a topical ointment. In one non-limiting example, the method 12 may include depositing 20 the composition 10 into 110, 12 oz. containers. The method 12 may further include utilizing 22 the composition 10 as a topical ointment on livestock wounds that are larger and/or located at easier-to-bandage or reach places on the animal. The topical ointment of the composition 10 may be directly applied 22 on livestock wounds using an applicator, brush or sanitized hand. Alternatively, the topical ointment of the composition 10 may be directly applied 22 to dressings/bandages whereafter the dressings/bandages are applied to the wound. The amount of topical ointment of the composition 10 to be applied to the livestock wound should be consistent with the size and severity of the wound. Particularly, application of the topical ointment of the composition 10 should thinly coat the wound and the surrounding affected areas of the animal. The method 12 may also include applying 24 the topical ointment of the composition 10 to the livestock wound at least once daily to effectively treat the wound until healed.

Still further shown in FIG. 1, another aspect of the method 12 of the present disclosure may include depositing 26 the composition 10 into spray bottle(s) to be applied as a topical spray. In one non-limiting example, the method 12 may include depositing 26 the composition 10 into 83, 16 oz. spray bottles. The method 12 may further include utilizing 28 the composition 10 as a topical spray on livestock wounds that are smaller and/or located at harder-to-bandage or reach places on the animal. The topical spray of the composition 10 may be directly applied 28 (e.g., sprayed) on livestock wounds. The amount of topical spray of the composition 10 to be applied to the livestock wound should be consistent with the size and severity of the wound. Particularly, application of the topical spray of the composition 10 should thinly coat the wound and the surrounding affected areas of the animal. The method 12 may also include applying 30 the topical spray of the composition 10 to the livestock wound at least twice daily to effectively treat the wound until healed.

The composition 10 and method 12 of the present disclosure are universally applicable to livestock of all ages, breeds and species, including horses, cattle, dairy, sheep, swine, poultry and other domesticated animals. Although the disclosure has been described and illustrated with respect to preferred aspects thereof, it is not to be so limited since changes, modifications, and combinations thereof may be made which are within the full intended scope of the disclosure.

What is claimed is:

1. A composition for treating wounds in livestock, consisting of the following ingredients:
honey;
colloidal silver;
coconut oil;
collagen peptides;
turmeric;
sugar;
salt; and
bromelain;
wherein the composition is adapted to treat wounds in livestock.

2. The composition of claim 1, wherein the ingredients consist of the following weight percentages:
honey at approximately 29 wt. %;
colloidal silver at approximately 19 wt. %;
coconut oil at approximately 18 wt. %;
collagen peptides at approximately 10 wt. %;
turmeric at approximately 12 wt. %;
sugar at approximately 5 wt. %;
salt at approximately 5 wt. %; and
bromelain at approximately 2 wt. %.

3. The composition of claim 1, wherein the ingredients consist of natural, non-toxic and/or organic ingredients.

4. The composition of claim 1, wherein the colloidal silver is at approximately 120 ppm.

5. The composition of claim 1, wherein the composition is a mixture of the ingredients.

6. The composition of claim 1, wherein the composition is adapted to treat wounds in livestock through a topical ointment or a topical spray.

7. The composition of claim 1, wherein the composition is adapted to treat wounds in livestock located on a variety of places on an injured animal.

8. The composition of claim 1, wherein the composition is adapted to treat wounds in livestock comprising:
inflammation;
scratches;
strangles;
flesh wounds;
bacterial infections;
viral infections; and
fungal infections.

9. The composition of claim 1, wherein the livestock comprises horses, cattle, dairy, sheep, swine, poultry or other domesticated animals.

10. The composition of claim 1, wherein the composition is adapted to treat wounds in livestock on a diverse population encompassing varying species, breeds and ages.

11. A composition for treating wounds in livestock, comprising:
honey at approximately 29 wt. %;
colloidal silver at approximately 19 wt. %;
coconut oil at approximately 18 wt. %;
collagen peptides at approximately 10 wt. %;
turmeric at approximately 12 wt. %;
sugar at approximately 5 wt. %;
salt at approximately 5 wt. %; and
bromelain at approximately 2 wt. %;
wherein the composition is adapted to treat wounds in livestock.

12. The composition of claim 11, wherein the ingredients comprise natural, non-toxic and/or organic ingredients.

13. The composition of claim 11, wherein the colloidal silver is at approximately 100 - 150 ppm.

14. The composition of claim 11, wherein the composition comprises a mixture of the ingredients.

15. The composition of claim 11, wherein the composition is adapted to treat wounds in livestock through a topical ointment or a topical spray.

16. A method of treating wounds in livestock, comprising:
preparing a composition for treating wounds in livestock, the composition consisting of the following ingredients:
a) honey;
b) colloidal silver;
c) coconut oil;
d) collagen peptides;
e) turmeric;
f) sugar;
g) salt; and
h) bromelain;
adding the ingredients into a mixing vessel;
mixing the ingredients in the mixing vessel to form the composition; and
applying the composition to a livestock wound;
wherein the composition is adapted to treat wounds in livestock.

17. The method of treating wounds in livestock of claim 16, wherein the ingredients consist of the following weight percentages:
a) honey at approximately 29 wt. %;
b) colloidal silver at approximately 19 wt. %;
c) coconut oil at approximately 18 wt. %;
d) collagen peptides at approximately 10 wt. %;
e) turmeric at approximately 12 wt. %;
f) sugar at approximately 5 wt. %;
g) salt at approximately 5 wt. %; and
h) bromelain at approximately 2 wt. %.

18. The method of treating wounds in livestock of claim 16, further comprising:
depositing the composition into container(s) to be utilized as a topical ointment; and
applying the topical ointment of the composition to a livestock wound at least once daily to effectively treat the wound.

19. The method of treating wounds in livestock of claim 16, further comprising:
depositing the composition after mixed into spray bottle(s) to be utilized as a topical spray; and
applying the topical spray of the composition to a livestock wound at least twice daily to effectively treat the wound.

20. The method of treating wounds in livestock of claim 16, wherein the ingredients are mixed in the mixing vessel for at least 20 minutes to form the composition.
